(19) United States
(12) Patent Application Publication
(10) Pub. No.: US 2016/0051412 A1
(43) Pub. Date: Feb. 25, 2016

(54) WOUND CARE MASK

(71) Applicant: Ansell Limited, Richmond (AU)

(72) Inventors: Jeffrey C. Moreland, Simpsonville, SC (US); Roger E. Huckfeldt, Nixa, MO (US); Eric Thompson, Central, SC (US); Jamie Ashworth, Freehold, NJ (US); Kristin King, West New York, NJ (US); Carrie Lirae Merck, Clemson, SC (US)

(21) Appl. No.: 14/835,168

(22) Filed: Aug. 25, 2015

Related U.S. Application Data
(60) Provisional application No. 62/041,168, filed on Aug. 25, 2014.

(51) Int. Cl. A61F 13/00

(52) U.S. Cl. CPC A61F 13/00008

(57) ABSTRACT

A wound care mask, including a fabric knitted from a first yarn; at least one stress release dart knitted within the knitted fabric; and at least one flap having a fastener disposed thereon, wherein the at least one fastener is adapted to engage the knitted fabric to releasably tighten or loosen a fit to a head, is disclosed.

### CROSS-REFERENCE TO RELATED APPLICATIONS

This application claims the benefit of U.S. Provisional Application No. 62/041,168, filed on Aug. 25, 2014, which is incorporated herein in its entirety.

### BACKGROUND

1. Field of the Invention

Embodiments of the present invention generally relate to wound care articles and, more particularly, to wound care masks and skullcaps, and the like, optionally comprising moisture-wicking yarns and/or anti-microbial treatments and/or outer dressings. Methods of manufacturing the masks and skullcaps are also disclosed.

2. Description of the Related Art

Wounds, such as cuts, abrasions, and burns and chronic wounds, such as lesions and ulcers, ulcers caused by dermatitis, contact dermatitis, vasculitis, psoriasis, diabetes, and the like, require constant care. Also, bed ridden people may suffer from pressure ulcers, i.e., as a consequence of paralysis. Treatment of these many types of wounds poses unique problems for medical personnel. On one hand, moisture and air are required to allow wounds to heal for most but not all wounds. However, the healing of some wounds, particularly chronic wounds, can be slowed by the presence of too much moisture, such as wound exudate. Also, wounds of this type are acutely susceptible to infection and therefore must be insulated from germs, bacteria, viruses, and other pathogens, although monitoring the healing of wounds is performed during treatment.

Gauzes and other medical dressings, which are typically woven structures, have been used to treat cuts, abrasions, and chemical-, heat- and flame-caused burns and are highly absorbent, and therefore treatment entails multiple changes of dressings, which takes a lot of time and is further disfavored because changing dressings often means abrading the surface of the wound, delaying healing. Further still, some wounds require compression-type therapies, which consists of additional layer(s) of dressings. In addition, many wearable articles consist of rigid, stiff fabrics leading to stress and irritation during donning, usage, and doffing.

Therefore, the inventors have provided anti-microbial articles, such as masks and caps, that manage moisture, exudate, and perspiration, are flexible for a tight and comfortable fit during mobility, are donned and doffed easily, and promote healing by allowing intimate contact of the anti-microbial mask with the skin of the wearer without sticking to or abrading the skin/wound as well as absorbent and/or highly wicking outer barrier dressings that can be changed without contacting a wound.

### SUMMARY

Embodiments according to the invention include a mask and methods for making wearable, easy to don and doff anti-microbial masks and/or outer dressings substantially as shown in and/or described in connection with at least one of the figures, as set forth more completely in the claims. Various advantages, aspects, and novel features of the present disclosure, as well as details of an exemplary embodiment thereof, will be more fully understood from the following description and drawings.

### BRIEF DESCRIPTION OF THE DRAWINGS

So that the manner in which the above recited features of the present invention can be understood in detail, a more particular description of the invention, summarized above, may be had by reference to embodiments, some of which are illustrated in the appended drawings. It is to be noted that the appended drawings illustrate typical embodiments of this invention and are not to be considered limiting of its scope, for the invention admits to other equally effective embodiments. It is to be understood that elements and features of one embodiment may be in other embodiments without further recitation. Also, where possible, identical reference numerals have been used to indicate comparable elements common to the figures.

FIG. 1 depicts a flattened, unsewn view of a wound care mask in accordance with embodiments of the invention;

FIG. 2 depicts sewn view of the wound care mask of FIG. 1 in accordance with embodiments of the invention;

FIG. 3 depicts a front perspective view of the wound care mask of FIG. 2 wrapped around a head, in accordance with embodiments of the invention;

FIG. 4 depicts a back perspective view of the wound care mask of FIG. 2 wrapped around a head, in accordance with embodiments of the invention;

FIG. 5 depicts an outer dressing in accordance with embodiments of the invention;

FIG. 6 depicts a bottom perspective view of a skull cap outer dressing in accordance with embodiments of the invention; and

FIG. 7 depicts a plan view of a yarn used in embodiments according to the invention.

### DETAILED DESCRIPTION

Embodiments of the present invention comprise anti-microbial masks comprising a first yarn that fits around the head of a patient. The anti-microbial mask has at least one flap integrally formed therewith, and optionally two, three, or four flaps. In this context, integrally formed indicates that the mask and at least one flap comprises, optionally, a single piece of knitted fabric that is not two discrete pieces of fabric joined or adhered together, thereby avoiding a seam that might otherwise be a site for abrading skin. Embodiments of the invention comprise masks having flaps capable of adjusting compression around the head and/or neck placed therein, i.e., masks may be loose fitting and releasably tightened to view wounds, and the progress of healing, easily and without contacting the wound itself, and re-wrapping the head and/or neck without changing the mask.

At least one exemplary embodiment according to the invention comprises a wound care mask that includes a knitted fabric comprising a first metalized yarn having both wicking and antimicrobial characteristics, where the knitted fabric is formed to substantially conform to a head; at least one of a stress release dart knitted within the knitted fabric or at least one sewn ridge knitted within the knitted fabric; and at least one flap disposed and formed as part of the knitted fabric, wherein the at least one flap has a fastener adapted to engage the knitted fabric to releasably tighten or loosen a fit of the knitted fabric to the head.

Any embodiment according to the invention disclosed herein can promote the healing of wounds, such as chronic wounds, e.g., Stage I-IV dermal ulcers, diabetic ulcers, lesions and ulcers caused by dermatitis, contact dermatitis, psoriasis, etc., as well as patients suffering from pressure ulcers, burns, cuts, and/or abrasions.

FIG. 1 depicts a flattened, unsewn view 100 of a wound care mask 102 in accordance with embodiments of the invention. The wound care mask 102 is either a woven or knitted article and comprises a fabric having an anti-microbial and/or highly wicking yarn and may comprise a metalized yarn. The wound care mask 102 comprises a first half 130 and a second half 140. The first half 130 comprises a first upper arcuate edge 132, which corresponds to a forehead area of a head, a first back arcuate edge 134, corresponding with a backhead area of a head, a first lower arcuate area 136, corresponding with a neck area of a head, and a first lower arcuate area 138, which corresponds with a chin area when worn on a head of a patient. The second half 140 comprises a second upper arcuate edge 142, which corresponds to a forehead area of a head, a second back arcuate edge 144, corresponding with a backhead area of a head, a second lower arcuate area 146, corresponding with a neck area of a head, and a second lower arcuate area 148, which corresponds with a chin area when worn on a head of a patient. The first half 130 and the second half 140, which are optionally substantially symmetrical in embodiments according to the invention, meet in an integrally formed fabric knitted in the same knitting operation or, alternatively, are joined at a central point 150. Also, the second half 140 further comprises at least one flap 116, having at least one fastener 114, distal to the central point 150. As shown, the wound care mask 102 comprises two flaps 116 formed integrally therewith, which can be used to releasably tighten and loosen a fit of the wound care mask 102 around the head of a patient. Some embodiments according to the invention comprise two, three, four or more flaps 116, which can be used to tighten some parts of the wound care mask 102 while leaving a looser fit in other parts.

The wound care mask 102 further comprises darts 110 and 112 on the first half 130 and the second half 140 respectively. The darts 110 and 112 mate when the wound care mask 102 is folded along plane 108, which is generally near the central point 150, and are sewn together to form a stress release dart (as discussed in greater detail below). For example, the dart 110 has a front surface 111 and the dart 112 has a front surface 113, distal portions 111a and 113a, which are placed into contact with each other and sewn. The wound care mask 102 comprises the fasteners 114, such as hooks, for example, the hooks side of a VELCRO® fastener. As shown, the fasteners 114 are disposed on the flaps 116. The flaps 116 may be shorter or longer. In embodiments in which the flaps 116 are longer, the flaps 116 can go around half or more of a head and can be significantly tightened, creating a compression mask. When the flaps 116 are shorter, they travel a shorter distance around a head and may engage the wound care mask 100 less tightly. In either case, the loops side of a hook and loops fastener is optional. In other words, the fasteners 114 can engage the loops of a knitted article, such as the wound care mask 102, or the interstices of a woven article, and therefore the flaps 116 are capable of being as tight or loose as medically appropriate without consideration of a mating “loop” side fastener as is normally provided in a hooks and loops fastener.

The wound care mask 102 comprises fold points at a nose portion 104 (generally correlating with an area where a bridge of a nose would be if the mask were on a head) and a mouth portion 106 (generally correlating to an area where a mouth would be). The wound care mask 102 is folded along a plane 108 running through the wound care mask 102. At least one metalized yarn according to embodiments of the invention comprises a nylon yarn having a metal infused or otherwise coated thereon or therein, such as elemental silver, gold, zinc, copper, iodine, and/or their oxides, and/or alloys, and/or a noble metal-ion. Other examples according to embodiments of the invention comprise a silver-coated nylon 6,6 yarn, such as yarns manufactured by Noble Biomaterials of Scranton, Pa., such as an X-static® brand yarn. Alternatively, the fabric comprises a yarn manufactured by Iftna, Inc. of Ontario, Canada. Any wound care mask or outer dressing discussed herein may be knitted using a covered yarn, as discussed below, and any wound care mask or outer dressing may be a 10, 13, 15 or 18 gauge knitted structure.

At least one exemplary embodiment according to the invention comprises a wound care mask 102 that includes a knitted fabric having a first metalized, highly wicking yarn, at least one stress release dart knitted within the knitted fabric; at least one sewn ridge knitted within the knitted fabric, typically corresponding with an area traversing from an upper forehead/frontal area to a parietal area; and at least one flap disposed and integrally formed within the knitted fabric, wherein the at least one flap has a fastener adapted to engage the knitted fabric to releasably tighten or loosen a fit to a head. In some embodiments, the fastener comprises hooks, such as the “hooks” side of a hooks and loops fastening system, e.g., VELCRO®.

Also, at least one exemplary embodiment according to the invention is a wound care mask 102 comprising a knitted fabric that includes a yarn having both highly wicking and antimicrobial characteristics, where the fabric is formed to substantially conform to a head; at least one flap disposed and formed as part of the knitted fabric, wherein the at least one flap has a fastener adapted to engage the knitted fabric to releasably tighten or loosen a fit of the knitted fabric to a head.

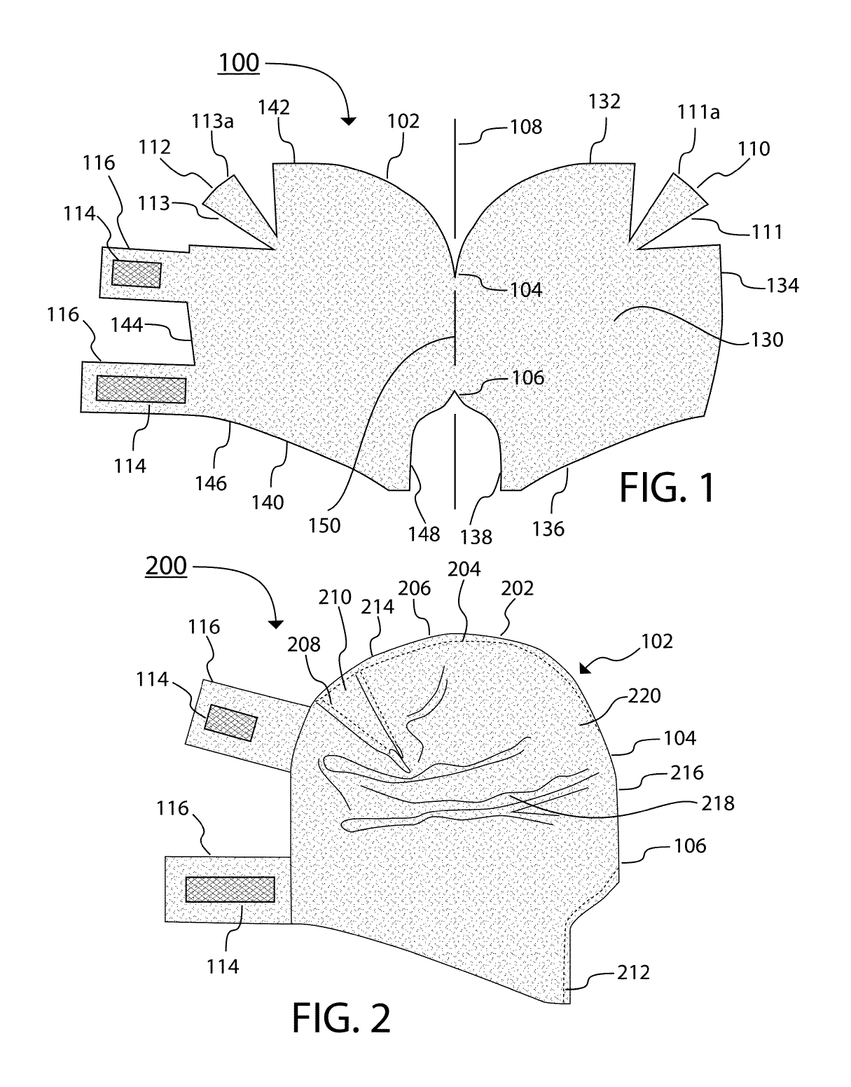

FIG. 2 depicts a sewn view 200 of the wound care mask 102 of FIG. 1, in accordance with embodiments of the invention. The wound care mask 102 is shown sewn with stitches 204 at an upper edge 202, creating a sewn ridge 206 that extends from a back of head portion 214 to a nose portion 104. The darts discussed above are shown sewn with stitches 208 to create a stress release dart 210. The wound care mask 102 is also sewn with stitches 212 near the mouth portion 106. A seamless front portion 216 is unsewn and extends between the nose portion 104 and the mouth portion 106. The wound care mask 102 comprises wrinkles 218 in what would be a temple area of a head. In other words, when the stress release dart 210 and the sewn ridge 206 are sewn, the fabric comprising the wound care mask 102 is bunched up, so that the wound care mask 102 is not flat. The wrinkles 218 may be in other areas of the wound care mask 102 using similar techniques, allowing a loose fitting that is easily put on and taken off the head of a patient without abrading a wound. The fasteners 114 having the hooks on the flaps 116 are shown outstretched, i.e., not engaged with any part of the wound care mask 102. However, when the fasteners 114 are engaged with another part of the wound care mask 102, the fasteners 114 can be as tight or as loose, without having the loops portion of a fastener in a specific area, as appropriate for any particular medical situation. For example, the flaps 116 are optionally long enough to traverse at least half way around the head of a patient and engage the wound care mask 102 in any area, so many sizes, fits, and tensions/compressions for different patients or, also, for the same patient as swelling recedes, may be accommodated. Moreover, because the wound care mask 102 is wrapped around a head of a patient, it can be as tight or as loose as needed and simultaneously maintain contact with a wound.

The wound care mask 102 comprises a single or multi-piece construction of fabric. Furthermore, it can be easily customized for specific medical conditions. In other words, the fabric can be cut out at, for example, the nose portion 104 to allow easier breathing to the patient. Alternatively, the nose portion 104 may not be cut out if the nose itself exhibits a wound. Also, because the size and location of noses from person to person varies, the wound care mask 102 is further customizable, i.e., cutouts are made where and as large as medically appropriate. Similarly, cutouts may be made in the mouth portion 106, an eye area 220, an ear area 222, or any other area where it is desirable to do so, for example, because an area needs to be ventilated or there is no wound. At least one embodiment according to the invention comprises a wound care mask 102 that is knitted and cut and sewn to a shape adapted to be fit or wrapped around a head and comprising a silver-coated nylon yarn, such as the X-static® yarn disclosed above. Furthermore, a knitted and cut and sewn wound care mask 102 may comprise any yarn disclosed herein, including, for example, the incorporation of a plaited yarn.

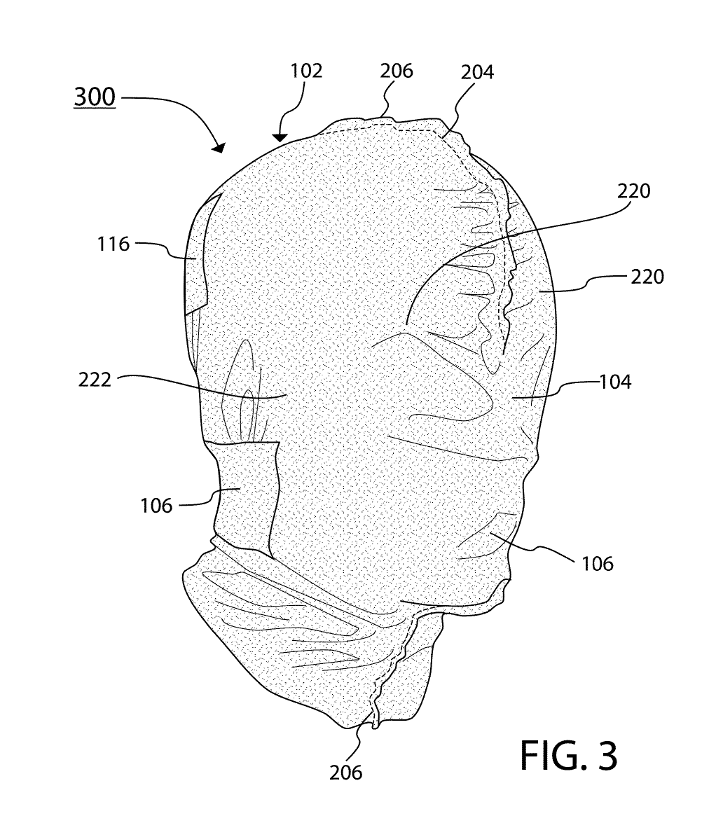

FIG. 3 depicts a perspective view 300 of the wound care mask 102 of FIG. 2 wrapped around a head, in accordance with embodiments of the invention. The perspective view 300 shows the wound care mask 102 from a front of head perspective. The wound care mask 102 is depicted as wrapped around a head and tightened thereto with the fasteners 114 on the flaps 116. As discussed above, the eye areas 220, the ear areas 222, the mouth portion 106, the nose portion 104, and any other area of the wound care mask 102 may be cut out.

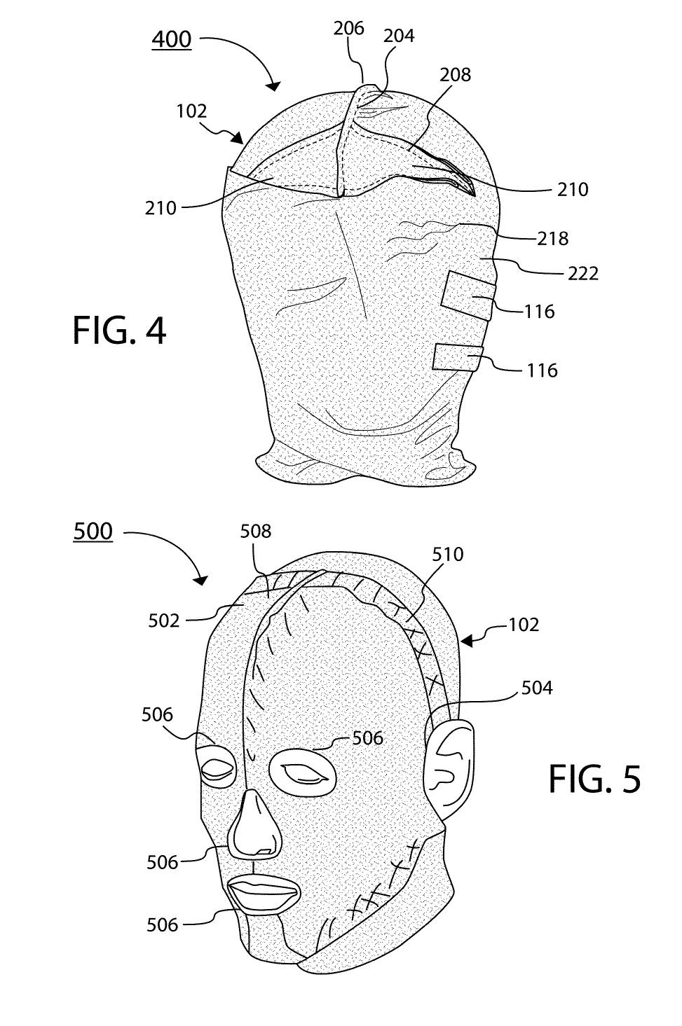

FIG. 4 depicts a perspective view 400 of the wound care mask 102 of FIG. 2 wrapped around a head, in accordance with embodiments of the invention. The perspective view 300 shows the wound care mask 102 from a back of head perspective. The wound care mask 102 is depicted as wrapped around a head and tightened thereto with the flaps 116. The stress release darts 210, sewn with the stitches 208 and the sewn ridge 206, sewn with the stitches 204, are also shown. As discussed above, because the wound care mask 102 is loose fitting and releasably tightened with the flaps 116, the wound care mask 102 may be removed to view wounds and the progress of healing, re-fitted easily, without contacting the wound itself.

Outer dressings, which are optionally placed over masks, in accordance with embodiments of the invention, are further contemplated herein. The wound care mask 102 can remain on the wound, for example a burn, for an extended period of time, while the outer dressing might be changed every day or as medically appropriate. A burn patient can also self-dress a wound easily and effectively once sent home from a hospital using either or both of the wound care mask 102 and/or outer dressing described below.

FIG. 5 depicts an outer dressing 500 in accordance with embodiments of the invention. The outer dressing 500 is for use with a wound care mask, such as the wound care mask 102, as discussed above. Embodiments according to the invention also comprise wherein the outer dressing 500 is a single seamless layer. The outer dressing 500 may comprise a single yarn having one, two, three, four or more filaments. For example, a single yarn may be one yarn. Alternatively, a single yarn may comprise a core yarn that has one or more yarns wrapped around the core yarn, i.e., a covered yarn. The outer dressing 500 may also have two seamless layers. For example, a first yarn that is plaited with a second yarn, i.e., two separate yarns that knitted into the same stitch with the same needle(s) at the same time would indicate a two layer seamless outer dressing. Embodiments of the invention further include a two-piece construction, having two halves, which are adhered to each other releasably with fasteners, such as hook-and-loop fasteners, such as VELCRO®. The outer dressing 500 comprises a front half 502 and a rear half (not shown). The front half 502 of the outer dressing 500 comprises hook fasteners (not shown) on the underside of a perimeter 510. The hook fasteners on the perimeter 510 may engage a loops aspect of a fastener located on the rear half of the wound care mask 102 or, alternatively, may engage and be secured with the loops or interstices of the wound care mask 102 itself. A two-piece construction may be favorable in some applications because the inherent seams of a two-piece (or more than two-piece) construction are not critical because the seams will not directly contact the skin/wound of a patient. Moreover, a two-piece outer dressing may be easier to remove and/or replace in conjunction with treatment for burns and/or other wounds, particularly more serious burns or wounds.

Also, the front half 502 further comprises a sewn ridge 508, which can accommodate the ridge of the wound care mask, as discussed above. The front half 502 may further comprise cuts outs 506 in various areas, as needed. Embodiments further include a half cutout 504, such as in an ear area on the front half 502 while a corresponding cutout 504 (not shown) may be present on the rear half of a two-piece outer dressing. In at least one embodiment of the invention, the outer dressing comprises a one-piece construction substantially similar to the wound care mask 102. In other words, the outer dressing 500 is optionally designed to have darts that are similar to the darts 110 and 112 and the fasteners 114 located on the flaps 116 of the wound care mask 102 as well as a seamless construction located between a nose area and a mouth area. The flaps 116 are optionally long enough to traverse at least half way around the head of a patient, so the flaps can accommodate many sizes, fits, and tensions/compressions for different patients or, also, for the same patient as swelling around a wounded area recedes. At least one wound care mask according to the embodiments of the invention comprises at least three flaps, wherein a first flap is wrapped tightly around a head, a second flap is wrapped more tightly than the first flap, and a third flap is wrapped more tightly than the second flap, creating a pressure gradient from the first flap to the third flap. In all wound care masks discussed herein, the amount of compression can be varied as needed.

The outer dressing 500 may be a knitted article or a woven fabric, substantially similar to articles disclosed herein, including the incorporation of silver, silver alloys, or other metals in yarns for anti-microbial effects. In some embodiments, the outer dressing 500 is a 10-18 gauge knit. In some embodiments, it is a 13 gauge knit. Where a denser outer dressing is desirable, such as to absorb greater amounts of moisture, embodiments of the invention comprise an 18 gauge knit. In some embodiments, the yarn used to manufacture any skin-contacting article discussed herein and/or outer dressing 500 is a silver-coated nylon 6,6 yarn, which is, for example, between 140 and 221 denier, which is capable of being knit with an 18 gauge needles, such as an X-static® yarn manufactured by Noble Biomaterials of Scranton, Pa. In at least one embodiment according to the invention, a 13 gauge or 15 gauge needle(s) is used, for wound care masks for treating certain wounds requiring exposure to oxygen for healing. Without intending to be bound by theory, it is believed that a 13 gauge or 15 gauge knitted structure, which has larger interstices between the courses of yarn loops, which may be used for knitting yarns from 300 to 600 denier or larger, allows greater amounts of oxygen to reach wounds covered therewith. Alternatively, the skin-contacting article comprises a fabric containing an anti-microbial manufactured by Iftna, Inc. of Ontario, Canada. The outer dressing 500 may also comprise a cotton fiber, rayon, a rayon/cotton blend, a polyethylene filament, or the like, releasably placed over the wound care mask. An outer dressing 500 having cotton provides moisture absorption while the rayon is lubricious so that the outer dressing 500 slides easily over the wound care mask discussed above.

The outer dressing 500, which may be knit or woven, further comprises a highly elastic yarn, such as SPANDEX® or LYCRA®, so that, when the outer dressing is stretched during use, it clamps onto the primary article, i.e., a wound care mask, which holds the wound care mask in place and tightly to the skin of a patient. As above, regarding the wound care mask, the outer dressing 500 may comprise highly-wicking yarns, including, for example, STA-COOL® polyester, ringspun hydrophilic polyester HYDROTEC®, or DRYENERGY® polyester/cotton and may further comprise any shape for the treatment of a limb or body part, such as a leg, arm, knee, ankle, head, waist, and the like. Micro-denier, multi-filament yarns, and yarns having non-circular, irregularly shaped cross-sections are particularly effective at wicking moisture away from one area, such as a wound, to other areas. Also, embodiments of the invention include wherein the outer dressing 500 and the wound care mask 102 comprise a silver plated nylon, so that the outer dressing 500 is of a type that is a barrier against germs encountered from the external environment while the wound care mask 102, which contacts the skin, is a type of barrier that can inhibit germs emanating from within the body of a patient.

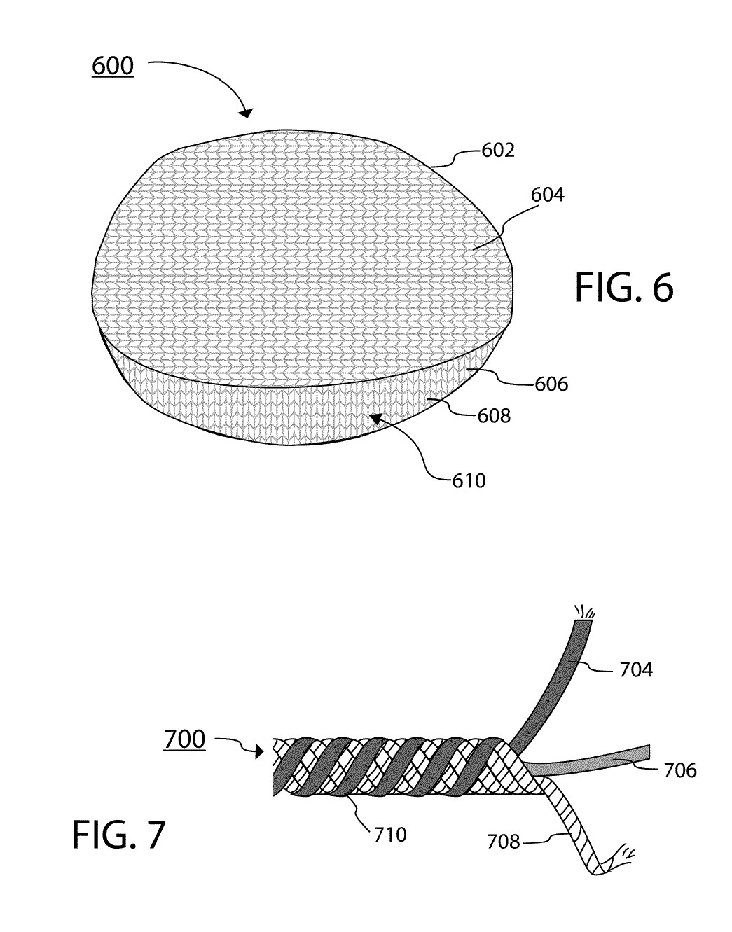

FIG. 6 depicts a bottom perspective view of a skull cap outer dressing 600 in accordance with embodiments of the invention. The skull cap outer dressing 600 comprises a one-piece seamless knitted wound care article in the style of a dome or a “beanie.” The skull cap outer dressing 600 is knitted, for example, weft-knitted from a yarn so that horizontal courses 604 disposed on the external surface 602 have corresponding vertical courses 608 disposed on an internal surface 606. Because the vertical courses 608 are disposed on a skin contacting interior portion 610, which can be placed on a head of a patient, the skull cap outer dressing 600 slides more easily over, for example, the wound care mask 102 discussed above, and therefore avoiding abrading the wound because there is less movement when the skull cap outer dressing 600 contacts the wound care mask 102. Also, the skull cap outer dressing 600 can be knitted with, for example, a silver-coated nylon yarn or a covered yarn, for example, a yarn having an elastomeric yarn as a core that is covered with a cotton yarn which is covered with a nylon yarn.

Also, at least one embodiment according to the invention includes a yarn having an elastomeric yarn as a core that is covered with a cotton yarn which is covered with a nylon yarn and has an additional wrapping yarn that comprises a silver-coated nylon yarn. Furthermore, in some embodiments according to the invention, the skull cap outer dressing 600 comprises a yarn having an elastomeric yarn as a core that is covered with a cotton yarn which is covered with a nylon yarn and which is knitted with a plaited yarn that further comprises a silver-coated nylon yarn. Moreover, the skull cap outer dressing 600 may be knitted with any yarn disclosed herein. As discussed below, the skull cap outer dressing 600 comprises yarns that are absorbent, highly wicking and fluid managing, and provide anti-microbial barrier properties as well as providing a snug and flexible fit.

FIG. 7 depicts a plan view of a yarn 700 used in embodiments according to the invention. The yarn 700 comprises a covered yarn. For example, an elastomeric yarn or filament 706, such as SPANDEX® OR LYCRA®, is a core for the yarn 700. The elastomeric yarn or filament 706 has an absorbent fiber, yarn, or filament 708, such as cotton, wrapping the elastomeric yarn or filaments 706. As shown, the absorbent fiber, yarn, or filament 708 is twisted. The absorbent fiber, yarn, or filament 708 is then covered with a nylon yarn 710 for moisture management properties. Optionally, the nylon yarn 710, which most closely contacts the skin, further comprises an anti-microbial coating 704. The anti-microbial coating 704 may be any of the anti-microbial coatings discussed herein. Embodiments of the yarn 700 include wherein the anti-microbial coating 704 is elemental silver, which, in addition to providing anti-microbial properties, also promotes healing of wounds. Also, at least one embodiment according to the invention comprises wherein the yarn 700 comprises a second yarn (not shown), such as an additional nylon yarn that covers the yarn 700, and having an anti-microbial coating 704 disposed thereon as an external covering.

The following yarn and/or knitting structures are capable of achieving absorbency (cotton), stretch (elastomeric yarn), and wound healing/anti-microbial (silver coating) and moisture management (highly wicking nylon yarns) within a single layer, any of which may be used to knit a mask or an outer dressing, as discussed further below, therefore permitting the masks and/or outer dressings made therewith to be thin and flexible as well as absorbent and highly wicking. At least one yarn comprises a core having a 354 denier (dn) (30/2CC) cotton and 40 dn elastomeric yarn having a covering that comprises a 70 dn nylon 6,6, optionally further comprising elemental silver coated thereon. At least one yarn comprises a core having 354 dn (30/2CC) cotton and a 40 dn elastomeric yarn, and a first cover comprising 70 dn nylon 6,6 and a second cover yarn comprising a 70 dn silver coated nylon, such as an X-Static yarn. Also, at least one outer dressing and/or masks and/or knitted article, such as a sock or compression sleeve, comprises a yarn having a core comprising 354 dn (30/2CC) cotton, and a 40 dn elastomeric yarn, and a cover yarn comprising 70 dn nylon 6,6, which is knitted with a plating yarn comprising a 70 dn silver coated nylon yarn, e.g., X-Static nylon.

The wound care mask 102 optionally further comprises a moisture wicking fiber or yarn that promotes the wicking of moisture from one area, such as a wound, to an outer dressing. Also, the anti-microbial mask comprises a second layer to transport and absorb moisture, such as perspiration, wound exudate, and the like. The wound care mask 102 may be knitted by conventional knitting processes and may further comprise various deniers of yarns and knitted using needles of appropriate gauges for a given denier, which can be used to increase the channeling efficiency and distribution of moisture from one area of the article to other areas and to the second layer. In other words, a first layer may be disposed as a skin-contacting layer while a second layer, disposed over the first layer, is further away from the skin of a patient. Embodiments of the invention can manage moisture, i.e., keep a wound moist but not wet, which can both inhibit bacterial growth and/or manage toxins emanating from the wound itself. Embodiments of the invention can wick moisture in all three axes, i.e., around the circumference of a head or neck, along a longitudinal axis of a head or neck, i.e., from the chin towards the eyes, and/or from a surface of skin to, for example, a first layer (skin-contacting) of the wrap and, optionally, to a second layer, e.g., an outer dressing. Embodiments according to the invention comprise additional knitted layers.

The wound care mask 102 may comprise more than one yarn. In some embodiments of the invention, a first yarn is an absorbent, highly moisture-wicking yarn having an anti-microbial coating for the treatment of wounds and the second yarn comprises cotton, polyesters, nylons, or an elastic yarns, such as SPANDEX® or LYCRA®, and the like and/or any combination of these yarns. Cotton may be used, for example, to absorb and wick moisture, although nominally compared with the absorbent, highly moisture-wicking yarns disclosed herein. Elastic yarns, such as SPANDEX® or LYCRA®, may impart stretch and comfort properties. Moreover, masks can be specified to use specified amounts of elastic yarns that can enhance stretching of the mask, so that one size may fit all users and/or can allow easy tightening and loosening as needed for specific wounds. In at least one embodiment of the invention, the yarn comprises approximately 87% of a highly-wicking nylon yarn or filament having elemental silver coated thereon and 13% of an elastic yarn, which together act as a fluid managing, anti-microbial barrier, which may be twisted together to form a single yarn. Moreover, medical personnel can loosen the wound care mask 102, view the healing of a wound, and re-tighten the wound care mask 102 repeatedly without abrading the wound or changing the wound care mask 102.

In some embodiments, the mask comprises one layer, i.e., a first fabric layer, which contacts the skin of the wearer, and comprises metals, such as silver, gold, copper, or zinc, or their alloys or combinations of elemental metals and alloys or other substances having anti-microbial properties, which promotes healing of wounds and promotes an antiseptic environment. In some embodiments, the masks comprise a second, third, or fourth fabric layer disposed on the first layer, each comprising cotton, polyesters, nylons, elastic yarns, such as SPANDEX®, LYCRA®, and ELASTANE®; hydrophilic, and anti-microbial yarns. In some embodiments of the invention, a nylon yarn, for example, is used as a main yarn, while in other embodiments, nylon yarn may be plaited into a non-nylon main yarn. In other embodiments of the invention, two or more yarns are plaited throughout the article and, accordingly, the article has two layers throughout the entire article. The inner layer (a skin contacting layer) and outer yarns may optionally comprise the same nylon yarn, a different nylon, or a non-nylon yarn. Some yarns are treated with an anti-microbial agent, such as TRIOSYN®, triclosan, 2-propanol, quaternary ammonium compounds, n-halamines, or compounds and combinations thereof, for their anti-microbial properties. Silver-zinc and silver-copper zeolites are also suitable anti-microbials, as well as other anti-microbials known to those in the art, such as, but not limited to, polymeric biguanides, i.e., chlorhexidine gluconate.

In some embodiments, the mask comprises a hydrophilic yarn such as a nylon yarn, such as nylon 6,6 yarn, having irregularly shaped cross-sections for exceptional wicking properties, and significantly superior to cotton yarns, fibers or filaments. Nylons promote the transport of moisture from the wound to the outer layer. One such example of a yarn having an irregularly shaped cross-section for wicking properties is Nilit AQUARIUS®, which, in addition to wicking moisture and exudate from a wound more effectively and efficiently, also comprises more volume between strands, which can contain more of an anti-microbial agent. Also, micro-denier and multi-filament yarns also promote superior wicking action. Hydrophilic yarns allow the transfer of metal ions from the yarn to the wound or eluent, promoting healing of the wound. Non-stick yarns are also contemplated according to embodiments of the invention. For example, any of the yarns disclosed herein may be blended or covered or otherwise incorporate low surface energy yarns, such as modified polytetrafluoroethylene yarns and/or polyethylenes. It is to be noted that any yarn discussed herein may comprise a hemostatic fiber therein. For example, a hemostatic fiber, i.e., a fiber causing blood to coagulate, such as treated cotton, may be employed on a covering yarn that contacts the wound.

Embodiments of the invention may further comprise wherein the wound care mask 102 or the outer dressing 500 is knitted in accordance with the Knitted Variable Stitch Design (KVSD) and/or three-dimensional, Automatic-Knit-Liner technologies as is disclosed in commonly-assigned U.S. Pat. Nos. 6,962,064; 7,213,419; 7,246,509; 7,434,422; and 7,555,921, each of which is hereby incorporated by reference in its entirety. KVSD programming of knitted articles allows for areas of additional stretch, reinforcement, and building up of additional yarns in any area of the knitted structure, e.g., a mask. For example, varying the stitch dimensions, such as yarn tension and needle depth, can produce articles having different levels of stretchability. The tension of the yarn may be varied by adjusting the tension of the yarn between a pinch roller and a knitting head by computer control of a knitting machine, as is disclosed in commonly-assigned U.S. Pat. No. 7,434,422. A wound care mask 102 and/or an outer dressing 102 that is knitted tighter will have less stretchability. Varying the depth of penetration of the knitting needle into the article, and by casting off or picking up additional stitches in a knitted course, can also affect stretchability. A shallower needle penetration produces an article that is tighter and more difficult to stretch.

Furthermore, wound care masks 102 and outer dressings 500 can be knit with a knitting machine according to instructions provided via computer programming. Double-layered zones, i.e., plaited layers, for knitted articles may be formed using a variable plaiting process, increasing the stretch in key flex areas of the gloves by altering the number of plaited courses in each section, such as the knuckles or the crotch between the index finger and thumb. For example, stretchable multi-layer functional zones are formed by plaiting a second yarn, such as an elastic yarn, such as SPANDEX® or LYCRA®, every second course in areas of low flex of the knitted wound care mask 102 or the outer dressing 500. Furthermore, the flex in some areas may be increased by adding a different yarn every eighth course in sections where no second yarn was present. The use of every 4th and 8th course in the plaiting structure is for illustrative purposes only. The plaiting structure can range from every other course to every 9th course using machines, such as, but not limited to, models SFG-I, NSFG, and SWG, manufactured by Shima Seiki Mfg., Ltd.

The perspiration and moisture wicking properties of the wound care mask and outer dressing, such as the wound care mask 102 and outer dressing 500, may also be achieved as disclosed in commonly assigned U.S. patent application Ser. No. 13/538,368, which are incorporated herein by reference in entirety. This technology includes a super absorbent material comprised of an electrospun polyurethane and bound acrylate. One such super absorbent material is marketed as SNS Nanosorb® 28. SNS Nanosorb® 28 has a higher affinity for water compared with the nylon 6,6 yarns described herein. The super absorbent material pulls in moisture wicked to it by the nylon 6,6 yarn, which the super absorbent material subsequently pulls into its internal matrix, thereby acting as a moisture reservoir. Such moisture movement leaves the nylon 6,6 of the wound care mask 102, keeping the moisture away from a user's skin. Different thicknesses of the super absorbent material may be employed. Moreover, the super absorbent material can be used, in lieu of additional padding, to protect body parts from inadvertent bangs and bumps. Moisture absorption is enhanced, in embodiments of the invention, by including poly-acrylates, polyurethanes, polyvinyl alcohol, hydrogels, and other hydrophilic particles and/or materials, as are known to those in the art.

Embodiments of the invention comprise methods for treating wounds. At least one method for treating a wound, comprises wrapping a wound care that includes a first fabric layer around a head of a patient; and releasably adhering the wound care mask to itself with flaps having at least one fastener located on the flap, wherein the first fabric layer comprises an anti-microbial agent and a wicking yarn to promote wicking of moisture from a wound. The method optionally includes further comprising wrapping or disposing an outer dressing around the wound care mask. The method further comprises a first fabric layer that includes at least one of a metalized yarn, a highly wicking yarn, or a yarn that is both metalized and highly wicking, or a covered yarn, as discussed above. Also, the method further comprises wherein the first fabric layer includes an anti-microbial agent, wherein the anti-microbial agent is at least one of gold, copper, iodine, silver, or zinc or their alloys, a noble metal-ion, triclosan, 2-propanol, n-halamines, polymeric biguanides, quaternary ammonium compounds, chlorhexidine gluconate, silver-zinc and silver-copper zeolites, or compounds and combinations thereof.

It is further contemplated herein that the wound care mask 102 and the outer dressing can be sold as a kit. For example, a kit may comprise any wound care mask made of any yarn(s) discussed herein and an outer dressing for covering the wound care mask. The outer dressing may comprise, for example, at least one of cotton, rayon, or a blend of cotton and rayon. Furthermore, the outer dressing may comprise a first yarn and a second yarn plaited therewith, wherein the first yarn comprises an elastomeric core yarn wrapped with an absorbent cotton yarn wrapped with a metalized yarn and the second plaited yarn comprises a metalized nylon yarn.

Embodiments of the invention, discussed herein, are directed towards masks. It is to be further understood that other articles for wound care on other parts of the body are contemplated herein, such as hands, knees, elbows, ankles, and the like, which may contain all features of embodiments of the invention. Moreover, a wound care mask may be combined with a substantially cylindrical compression sleeve for the neck of a patient, as could be manufactured by knitting the two components together in a single knitting operation, to form an article that addresses more than one body part.

All ranges of numerical values for any dimension recited herein are exemplary, are not to be considered limiting, and include ranges therebetween, and can be inclusive or exclusive of the endpoints. Optional included ranges can be from integer values therebetween, at the order of magnitude recited or the next smaller order of magnitude. For example, if the lower range value is 0.1, optional included endpoints can be 0.2, 0.3, 0.4 . . . 1.1, 1.2, and the like, as well as 1, 2, 3 and the like; if the higher range is 8, optional included endpoints can be 7, 6, and the like, as well as 7.9, 7.8, and the like.

While the foregoing is directed to embodiments of the invention, other embodiments of the invention may be devised without departing from the scope thereof, and the scope thereof is determined by the following claims.

1. A wound care mask, comprising:
a knitted fabric comprising a first metalized yarn having both wicking and antimicrobial characteristics, where the knitted fabric is formed to substantially conform to a head;
at least one of a stress release dart knitted within the knitted fabric or at least one sewn ridge knitted within the knitted fabric; and
at least one flap disposed and formed as part of the knitted fabric,
wherein the at least one flap has a fastener adapted to engage the knitted fabric to releasably tighten or loosen a fit of the knitted fabric to the head.

2. The wound care mask of claim 1, wherein the first metalized yarn comprises at least one of silver, gold, or copper and/or oxides and/or alloys thereof.

3. The wound care mask of claim 1, wherein the first metalized yarn comprises nylons, cotton, polyesters, rayon, or any combination or blend thereof.

4. The wound care mask of claim 1, wherein the first metalized yarn comprises a covered yarn.

5. The wound care mask of claim 4, wherein the first metalized yarn further comprises at least one of an irregularly shaped cross section of a nylon 6 or a nylon 6,6 yarn.

6. The wound care mask of claim 4, wherein the covered yarn comprises an elastomeric yarn core wrapped with a cotton yarn that is wrapped with a metalized nylon yarn.

7. The wound care mask of claim 1, further comprising a second metalized yarn plaited with the first metalized yarn.

8. The wound care mask of claim 1, further comprising a plurality of flaps, having at least one fastener per flap, disposed on a backhead area.

9. The wound care mask of claim 1, further comprising an anti-microbial agent disposed thereon.

10. The wound care mask of claim 9, wherein the anti-microbial agent is at least one of triclosan, 2-propanol, n-halamines, polymeric biguanides, quaternary ammonium compounds, chlorhexidine gluconate, silver-zinc and silver-copper zeolites, or compounds and combinations thereof.

11. The wound care mask of claim 1, further comprising a moisture reservoir fluidly coupled with the single piece of fabric.

12. The wound care mask of claim 11, wherein the moisture reservoir comprises an electrospun polyurethane and bound acrylate.

13. The wound care mask of claim 11, wherein the moisture reservoir comprises at least one of polyacrylates, polyvinyl alcohol, or other hydrogel or hydrophilic particles.

14. A kit, comprising:
the wound care mask of claim 1; and
an outer dressing for covering the wound care mask.

15. The kit of claim 14, wherein the outer dressing comprises at least one of cotton, rayon, or a blend of cotton and rayon.

16. The kit of claim 14, wherein the outer dressing comprises a first yarn and a second plaited yarn.

17. The kit of claim 16, wherein the first yarn comprises an elastomeric core yarn wrapped with an absorbent cotton yarn wrapped with a metalized yarn and the second plaited yarn comprises a metalized nylon yarn.

18. A method for treating a wound, comprising:
wrapping a wound care mask having a first fabric layer around a head of a patient; and
releasably adhering the wound care mask to itself with flaps located on the wound care mask,
wherein the first fabric layer yarn includes an anti-microbial agent and a wicking yarn to promote wicking of moisture from a wound.

19. The method of claim 18, further comprising wrapping or disposing an outer dressing around the wound care mask.

20. The method of claim 18, wherein the anti-microbial agent is at least one of gold, copper, iodine, silver, or zinc or their alloys, a noble metal-ion, triclosan, 2-propanol, n-halamines, polymeric biguanides, quaternary ammonium compounds, chlorhexidine gluconate, silver-zinc and silver-copper zeolites, or compounds and combinations thereof.
